(19) United States
(12) Patent Application Publication
(10) Pub. No.: US 2017/0065608 A1
(43) Pub. Date: Mar. 9, 2017

(54) PROPHYLACTIC OR THERAPEUTIC AGENT FOR NEUROPATHIC PAIN ASSOCIATED WITH GUILLAIN-BARRE SYNDROME

(71) Applicants: KYUSHU UNIVERSITY, Fukuoka-shi (JP); NIPPON CHEMIPHAR CO., LTD., Tokyo (JP)

(72) Inventors: Toshiyasu IMAI, Misato-shi (JP); Toru KAWASAKI, Misato-shi (JP); Toru OGAWA, Misato-shi (JP); Kazuhide INOUE, Fukuoka-shi (JP)

(21) Appl. No.: 15/337,384

(22) Filed: Oct. 28, 2016

Related U.S. Application Data
(63) Continuation of application No. 14/119,223, filed on Feb. 18, 2014.

(30) Foreign Application Priority Data
May 25, 2011 (JP) 2011-116965

(51) Int. Cl. A61K 31/551; A61K 31/4525

(52) U.S. Cl. CPC A61K 31/551; A61K 31/4525

(57) ABSTRACT

A P2X₄ receptor antagonist such as paroxetine, a diazepinedione derivative having the following formula (IX) is used as an agent for preventing or treating neuropathic pain associated with Guillain-Barré syndrome:

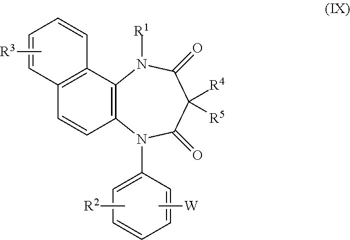

wherein R¹ is hydrogen, a C₁₋₈ alkyl group, or the like;
each of R² and R³ is hydrogen, a C₁₋₈ alkyl group, or the like;
each of R⁴ and R⁵ is hydrogen or the like; and
W is a five-membered or six-membered heterocyclic ring optionally having one or more substituents and comprising one to four nitrogen atoms as the members of the ring.

### FIELD OF THE INVENTION

The present invention relates to an agent for pre-venting or treating neuropathic pain associated with Guillain-Barré syndrome.

### BACKGROUND OF THE INVENTION

Guillain-Barré syndrome (GBS) is a peripheral neuropathy causing an acute motor paralysis. It has been known that the crisis of GBS usually follows an inspiratory or digestive infection.

In the past, GBS has been considered to be a demyelinating polyneuropathy, which attacks peripheral nervous myelin. It has recently been recognized that an axonopathy type results in a primary axonopathy.

Further, GBS is a monophasic disease, and its typical symptom is weakened limb muscles. Sensory disorders including dysesthesia often occur, and nearly 90% of patients complain of pains such as nerve root pain, muscle pain, joint pain or the like. GBS may cause cranial neuropathies such as facial paralysis, ocular motor paralysis, and swallowing or articulation disorders. At the climax phase, GBS may cause such a respiratory muscle paralysis that the patient should use a respirator, and it may also cause a severe autonomic neuropathy including hypertension, hypotension, fluctuations in blood pressure, tachycardia, or bradycardia.

While recovery starts after an acute phase, pain may continue from the acute phase to a recovery phase. At the recovery phase, the pain is an obstacle to rehabilitation. In the past, steroids, carbamazepine, opioid, gabapentin or the like have been used for the pain at the recovery phase in a supportive care. However, the obtained analgesic effect is often insufficient.

Guillain-Barré syndrome (GBS) is a peripheral neuropathy causing an acute motor paralysis. It has been known that the crisis of GBS usually follows an infectious disease. In the past, it has been considered to be a demyelinating polyneuropathy, which attacks peripheral nervous myelin. It has been recognized that an axonopathy type results in an axonopathy.

GBS is an autoimmune disease, and its relation with each of cellular immunity and humoral immunity has been suggested in reports. It is thought that the infectious disease prior to the crisis of GBS plays an important role.

The crisis of GBS is thought to be at one to two cases per 100,000 people annually. It is observed in all the generations, and male patients are slightly more than female ones.

GBS is a monophasic disease, and its typical symptom is weakened limb muscles. Sensory disorders including dysesthesia often occur, and nearly 90% of patients complain of pains such as nerve root pain, muscle pain, joint pain or the like. At the climax phase, GBS may cause such a respiratory muscle paralysis that the patient should use a respirator, and its case may be a severe autonomic neuropathy including hypertension, hypotension, fluctuations in blood pressure, tachycardia, or bradycardia. Therefore, a systemic management is very important at an acute phase. While recovery starts after an acute phase, Pain may continue from the acute phase to a recovery phase. At the recovery phase, the pain is an obstacle to rehabilitation. Steroids, carbamazepine, opioid, gabapentin or the like have been used for the pain in a supportive care. However, the obtained analgesic effect is often insufficient.

The present inventors have found that paroxetine, a diazepinedione derivative or the like having a P2X₄ receptor antagonism can be used as an agent for preventing or treating neuropathic pain, and filed patent applications (Patent documents 1 and 2).

The patent documents, however, do not clearly describe that the above-mentioned compounds are available as an agent for preventing or treating neuropathic pain associated with Guillain-Barré syndrome.

### PRIOR ART DOCUMENTS

### Patent Documents

Patent document 1: WO 2008/020651

Patent document 2: WO 2010/093061

### SUMMARY OF THE INVENTION

### Problems to be Solved by the Invention

It is the object of the invention to provide an agent for preventing or treating neuropathic pain associated with Guillain-Barré syndrome.

### Means for Solving the Problems

The present inventors have found that P2X₄ receptor antagonist such as paroxetine, a diazepinedione derivative or the like can be used as an agent for preventing or treating neuropathic pain associated with Guillain-Barré syndrome, and completed the present invention.

The present invention relates to an agent for preventing or treating neuropathic pain associated with Guillain-Barré syndrome containing P2X4 receptor antagonist as an active ingredient.

The invention also relates to an agent for preventing or treating neuropathic pain associated with Guillain-Barré syndrome containing a compound having the following formula (I) or a pharmacologically acceptable salt thereof as an active ingredient:

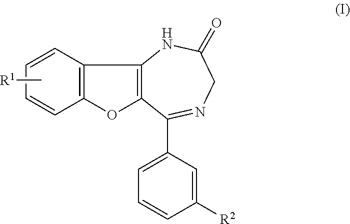

wherein R¹ is a halogen atom; and

R² is hydrogen, a halogen atom, nitro, cyano, —C(O)—OR³, —C(O)—NR⁴R⁵, —SO₂—OR³, or —SO₂—NR⁴R⁵, wherein each of R³, R⁴, and R⁵ is hydrogen or a C₁₋₆ alkyl group; or in the alternative

R¹ is hydrogen; and

R² is a halogen atom, nitro, cyano, —C(O)—OR³, —C(O)—NR⁴R⁵, —SO₂—OR³, or —SO₂—NR⁴R⁵, wherein each of R³, R⁴, and R⁵ is hydrogen or a C₁₋₆ alkyl group.

The invention further relates to an agent for preventing or treating neuropathic pain associated with Guillain-Barré syndrome containing a compound having the following formula (Ia) or a pharmacologically acceptable salt thereof as an active ingredient:

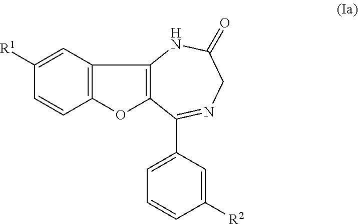

wherein R¹ is chloro or bromo; and

R² is hydrogen, chloro, bromo, nitro, or cyano; or in the alternative

R¹ is hydrogen; and

R² is chloro, bromo, nitro, or cyano.

The invention further relates to an agent for preventing or treating neuropathic pain associated with Guillain-Barré syndrome containing a compound having the following formula (II) or a pharmacologically acceptable salt thereof as an active ingredient:

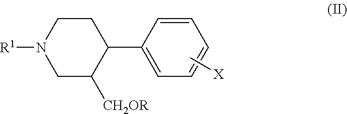

wherein R is a C₁₋₄ alkyl group, a C₂₋₄ alkynyl group, phenyl (optionally having one or more substituents selected from the group consisting of a lower alkyl group, an alkylthio group, an alkoxy group, a halogen atom, nitro, an acylamino group, methylsulfonyl, and methylenedioxy), or tetrahydronaphthyl;

R¹ is hydrogen; and

X is hydrogen, a C₁₋₄ alkyl group, a trifluoroalkyl group, hydroxyl, a halogen atom, methylthio, or an arylalkoxy group.

The invention further relates to an agent for preventing or treating neuropathic pain associated with Guillain-Barré syndrome containing a selective serotonin reuptake inhibitor as an active ingredient.

The invention further relates to an agent for preventing or treating neuropathic pain associated with Guillain-Barré syndrome containing an agent selected from the group consisting of imipramine, nortriptyline, amitriptyline, desipramine, doxepin, fluoxetine, fluvoxamine, citalopram, and a pharmacologically acceptable salt thereof as an active ingredient.

The invention further relates to an agent for preventing or treating neuropathic pain associated with Guillain-Barré syndrome containing a compound having the following formula (III) or a pharmacologically acceptable salt thereof as an active ingredient:

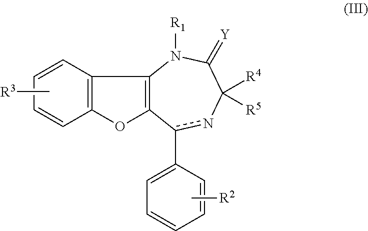

wherein X is S or CH₂;

Y is O, S, or NH;

R¹ is hydrogen, a C₁₋₈ alkyl group, a C₁₋₈ alkyl group having one or more halogen atoms, an aralkyl group comprising a C₁₋₆ alkyl moiety and a C₆₋₁₀ aryl moiety, a C₂₋₈ alkenyl group, carboxymethyl, or an alkoxycarbonylmethyl group comprising a C₁₋₈ alkoxy moiety;

each of R² and R³ independently is hydrogen, a C₁₋₈ alkyl group, a C₁₋₈ alkoxy group, a C₁₋₈ alkyl group having one or more halogen atoms, a C₁₋₈ alkoxy group having one or more halogen atoms, a halogen atom, amino, carboxyl, hydroxyl, nitro, cyano, a C₂₋₈ acyl group, a C₆₋₁₀ aryl group, or a five-membered or six-membered heterocyclic group;

each of R⁴ and R⁵ independently is hydrogen, a C₁₋₈ alkyl group, or a C₁₋₈ alkyl group having one or more halogen atoms; and

the double line consisting of a broken line and a solid line is a single bond or a double bond.

The invention further relates to an agent for preventing or treating neuropathic pain associated with Guillain-Barré syndrome containing a compound having the following formula (IV) or a pharmacologically acceptable salt thereof as an active ingredient:

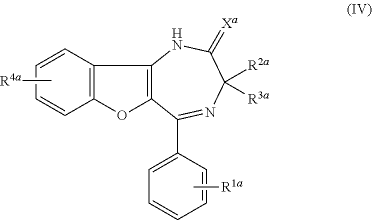

wherein X^{a} is O, S, or NH;

R^{1a} is hydroxyl, tetrazolyl, N(R^{5a}) (R^{6a}), a C₂₋₈ alkenyl group, a C₂₋₈ alkynyl group, a C₁₋₈ alkyl group having one or more halogen atoms, a C₁₋₈ alkoxy group having one or more halogen atoms, or a C₆₋₁₀ aryl group, wherein R¹ is hydrogen or a C₁₋₈ alkyl group, and R^{6a} is hydrogen, a C₁₋₈ alkyl group, or a C₂₋₈ acyl group;

each of R^{2a} and R^{3a} independently is hydrogen, a C₁₋₈ alkyl group, or a C₁₋₈ alkyl group having one or more halogen atoms; and

R^{4a} is hydrogen, a C₁₋₈ alkyl group, a C₁₋₈ alkoxy group, a C₁₋₈ alkyl group having one or more halogen atoms, a halogen atom, hydroxyl, nitro, amino, carboxyl, tetrazolyl, cyano, a C₆₋₁₀ aryl group, or a five-membered or six-membered heterocyclic group.

The invention further relates to an agent for preventing or treating neuropathic pain associated with Guillain-Barré syndrome containing a compound having the following formula (IVa) or a pharmacologically acceptable salt thereof as an active ingredient:

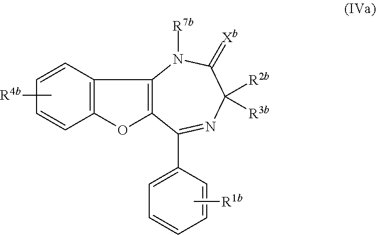

wherein X^{b} is O, S, or NH;

R^{1b} is a halogen atom, hydroxyl, tetrazolyl, N(R^{5b}) (R^{6b}), a C₂₋₈ alkenyl group, a C₂₋₈ alkynyl group, a C₁₋₈ alkyl group having one or more halogen atoms, a C₁₋₈ alkoxy group having one or more halogen atoms, or a C₆₋₁₀ aryl group, wherein R^{5b} is hydrogen or a C₁₋₈ alkyl group, and R^{6b} is hydrogen, a C₁₋₈ alkyl group, or a C₂₋₈ acyl group;

each of R^{2b} and R^{3b} independently is hydrogen, a C₁₋₈ alkyl group, or a C₁₋₈ alkyl group having one or more halogen atoms;

R^{4b} is hydrogen, a C₁₋₈ alkyl group, an alkoxy group, a C₁₋₈ alkyl group having one or more halogen atoms, a halogen atom, hydroxyl, nitro, amino, carboxyl, tetrazolyl, cyano, a C₆₋₁₀ aryl group, or a five-membered or six-membered heterocyclic group; and

R^{7b} is a C₁₋₈ alkyl group.

The invention further relates to an agent for preventing or treating neuropathic pain associated with Guillain-Barré syndrome containing a compound having the following formula (IVb) or a pharmacologically acceptable salt thereof as an active ingredient:

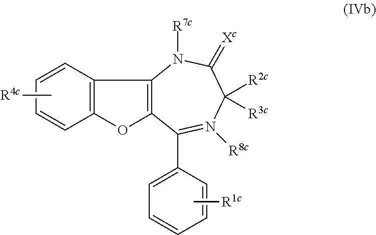

wherein X^{c} is O, S, or NH;

R^{1c} is hydrogen, a halogen atom, a C₁₋₈ alkyl group, a C₁₋₈ alkoxy group, hydroxyl, tetrazolyl, N(R^{5c})(R^{6c}), a C₂₋₈ alkenyl group, a C₂₋₈ alkynyl group, a C₁₋₈ alkyl group having one or more halogen atoms, a C₁₋₈ alkoxy group having one or more halogen atoms, or a C₆₋₁₀ aryl group, wherein R^{5c} is hydrogen or a C₁₋₈ alkyl group, and R^{6c} is hydrogen, a C₁₋₈ alkyl group, or a C₂₋₈ acyl group;

each of R^{2c} and R^{3c} independently is hydrogen, a C₁₋₈ alkyl group, or a C₁₋₈ alkyl group having one or more halogen atoms;

R^{4c} is hydrogen, a C₁₋₈ alkyl group, an alkoxy group, a C₁₋₈ alkyl group having one or more halogen atoms, a halogen atom, hydroxyl, nitro, amino, carboxyl, tetrazolyl, cyano, a C₆₋₁₀ aryl group, or a five-membered or six-membered heterocyclic group;

R^{7c} is hydrogen or a C₁₋₈ alkyl group; and

R^{8c} is hydrogen, a C₁₋₈ alkyl group, or a C₂₋₈ acyl group.

The invention further relates to an agent for preventing or treating neuropathic pain associated with Guillain-Barré syndrome containing a compound having the following formula (V) or a pharmacologically acceptable salt thereof as an active ingredient:

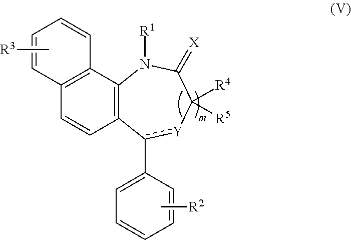

wherein X is O, S, or NH;

Y is N or NR⁶, wherein R⁶ is hydrogen or a C₁₋₈ alkyl group;

R¹ is hydrogen, a C₁₋₈ alkyl group, a C₂₋₈ alkenyl group, a C₁₋₈ alkyl group having one to three halogen atoms, or an alkyl group having phenyl;

R² is a C₁₋₈ alkyl group, a C₁₋₈ alkoxy group, a C₁₋₈ alkyl group having one to three halogen atoms, hydroxyl, nitro, amino, carboxyl, tetrazolyl, or cyano;

R³ is hydrogen, a C₁₋₈ alkyl group, a C₁₋₈ alkoxy group, a C₁₋₈ alkyl group having one to three halogen atoms, a halogen atom, hydroxyl, nitro, amino, carboxyl, tetrazolyl, or cyano;

each of R⁴ and R⁵ independently is hydrogen, a C₁₋₈ alkyl group, or a C₁₋₈ alkyl group having one to three halogen atoms;

m is 1 or 2;

when Y is N, the double line consisting of a solid line and a broken line is a double bond; and

when Y is NR⁶, the double line consisting of a solid line and a broken line is a single bond.

The invention further relates to an agent for preventing or treating neuropathic pain associated with Guillain-Barré syndrome containing a compound having the following formula (Va) or a pharmacologically acceptable salt thereof as an active ingredient:

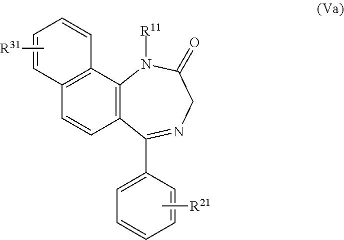

wherein R¹¹ is hydrogen or a C₁₋₈ alkyl group;

R²¹ is a C₁₋₈ alkyl group, a C₁₋₈ alkoxy group, a C₁₋₈ alkyl group having one to three halogen atoms, or hydroxyl; and

R³¹ is hydrogen or a halogen atom.

The invention further relates to an agent for preventing or treating neuropathic pain associated with Guillain-Barré syndrome containing a compound having the following formula (VI) or a pharmacologically acceptable salt thereof as an active ingredient:

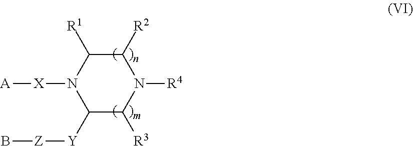

wherein A is an aryl group optionally having one or more substituents or a heterocyclic group optionally having one or more substituents;

B is an aryl group optionally having one or more substituents or a heterocyclic group optionally having one or more substituents;

X is a C₁₋₅ alkylene group or a bond;

Y is a C₁₋₅ alkylene group optionally comprising a double bond;

Z is O, S, N(R⁵), or a bond, wherein R⁵ is hydrogen or a C₁₋₈ alkyl group;

each of R¹, R², and R³ independently is hydrogen, a C₁₋₈ alkyl group, or a C₁₋₈ alkyl group having one to three halogen atoms;

R⁴ is hydrogen, a C₁₋₈ alkyl group, a C₁₋₈ alkyl group having one to three halogen atoms, a three-membered to seven-membered cycloalkyl group, or a C₁₋₈ alkyl group having a three-membered to seven-membered cycloalkyl group; and

each of n and m independently is 1 or 2;

provided that the substituent of the aryl group represented by A is not an alkyl group when X is a bond.

The invention further relates to an agent for preventing or treating neuropathic pain associated with Guillain-Barré syndrome containing a compound having the following formula (VIa) or a pharmacologically acceptable salt thereof as an active ingredient:

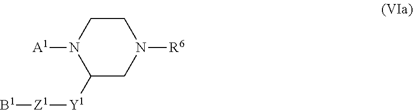

wherein A¹ is phenyl or thienyl, each of which optionally has one to three substituents selected from the group consisting of a halogen atom, a C₁₋₈ alkyl group having one to three halogen atoms, nitro, cyano, hydroxyl, amino, a C₁₋₈ alkylamino group, a C₂₋₁₆ dialkylamino group, a C₂₋₈ acylamino group, a C₁₋₈ alkoxy group, a C₁₋₈ alkoxy group having one to three halogen atoms, an aryl group, and a heterocyclic group;

B¹ is an aryl group optionally having one or more substituents or a heterocyclic group optionally having one or more substituents;

Y¹ is a C₁₋₅ alkylene chain optionally comprising a double bond;

Z¹ is O, S, N(R⁷), or a bond, wherein R⁷ is hydrogen or a C₁₋₈ alkyl group; and

R⁶ is hydrogen, a C₁₋₈ alkyl group, a C₁₋₈ alkyl group having one to three halogen atoms, or a three-membered to seven-membered cycloalkyl group.

The invention further relates to an agent for preventing or treating neuropathic pain associated with Guillain-Barré syndrome containing a compound having the following formula (VIb) or a pharmacologically acceptable salt thereof as an active ingredient:

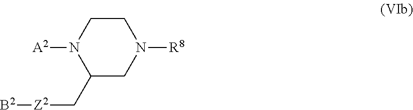

wherein A² is phenyl or thienyl, each of which optionally has one to three substituents selected from the group consisting of a halogen atom, a C₁₋₈ alkyl group having one to three halogen atoms, nitro, cyano, acetylamino, a C₁₋₈ alkoxy group, a C₁₋₈ alkoxy group having one to three halogen atoms, an aryl group, and a heterocyclic group;

B² is phenyl, naphthyl, benzofuranyl, 1,3-benzo[d]dioxolyl, quinolyl, indolyl, benzothienyl, thienyl, or pyridyl, each of which optionally has one to three substituents selected from the group consisting of a halogen atom, a C₁₋₈ alkyl group, a C₁₋₈ alkyl group having one to three halogen atoms, nitro, cyano, hydroxyl, amino, a C₂₋₈ acylamino group, a C₁₋₈ alkoxy group, a C₁₋₈ alkoxy group having one to three halogen atoms, a C₆₋₁₂ aryloxy group, sulfamoyl, a C₁₋₈ alkylsulfamoyl group, and a C₂₋₁₆ dialkylsulfamoyl group;

Z² is O, S, or NH; and

R⁸ is hydrogen or a C₁₋₈ alkyl group.

The invention further relates to an agent for preventing or treating neuropathic pain associated with Guillain-Barré syndrome containing a compound having the following formula (VII) or a pharmacologically acceptable salt thereof as an active ingredient:

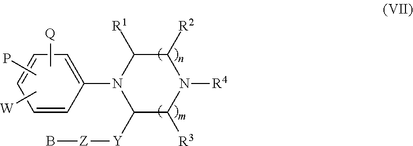

wherein B is an aryl group optionally having one or more substituents or a heterocyclic group optionally having one or more substituents;

Y is a C₁₋₅ alkylene group optionally comprising a double bond;

Z is O, S, N(R⁵), or a bond, wherein is hydrogen or a C₁₋₈ alkyl group;

each of R¹, R², and R³ independently is hydrogen, a C₁₋₈ alkyl group, or a C₁₋₈ alkyl group having one to three halogen atoms;

R⁴ is hydrogen, a C₁₋₈ alkyl group, a C₁₋₈ alkyl group having one to three halogen atoms, a three-membered to seven-membered cycloalkyl group, or a C₁₋₈ alkyl group having a three-membered to seven-membered cycloalkyl group;

each of P and Q independently is hydrogen, a halogen atom, a C₁₋₈ alkyl group, a C₁₋₈ alkyl group having one to three halogen atoms, nitro, cyano, hydroxyl, amino, a C₁₋₈ alkylamino group, a C₂₋₁₆ dialkylamino group, a C₂₋₈ acylamino group, a C₁₋₈ alkoxy group, a C₁₋₈ alkoxy group having one to three halogen atoms, or a heterocyclic group;

W is a C₁₋₈ alkyl group or a three-membered to seven-membered cycloalkyl group; or

when P and W are placed at 2- and 3-positions or 3- and 4-positions of phenyl, P and W are combined to form propylene or tetramethylene; and

each of n and m independently is 1 or 2.

The invention further relates to an agent for preventing or treating neuropathic pain associated with Guillain-Barré syndrome containing a compound having the following formula (VIII) or a pharmacologically acceptable salt thereof as an active ingredient:

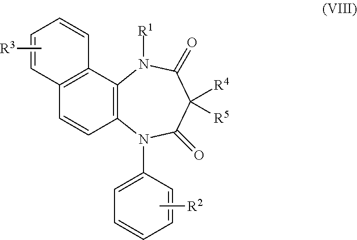

wherein R¹ is hydrogen, a C₁₋₈ alkyl group, a C₂₋₈ alkenyl group, a C₁₋₈ alkyl group having one to three halogen atoms, or a C₁₋₃ alkyl group having phenyl;

R² is hydrogen, a C₁₋₈ alkyl group, a C₁₋₈ alkoxy group, a C₁₋₈ alkyl group having one to three halogen atoms, a C₁₋₈ alkoxy group having one to three halogen atoms, a halogen atom, hydroxyl, nitro, cyano, amino, a C₁₋₈ alkylamino group, a C₂₋₈ dialkylamino group, a C₂₋₈ acylamino group, a C₂₋₈ acylamino group having one to three halogen atoms, a alkylsulfonylamino group, carboxyl, a C₂₋₈ acyl group, an alkoxycarbonyl group comprising a C₁₋₈ alkoxy moiety, carbamoyl, a C₁₋₈ alkylthio group, a C₁₋₈ alkylsulfinyl group, a C₁₋₈ alkylsulfonyl group, or sulfamoyl;

R³ is hydrogen, a C₁₋₈ alkyl group, a C₁₋₈ alkoxy group, a C₁₋₈ alkyl group having one to three halogen atoms, a C₁₋₈ alkoxy group having one to three halogen atoms, a halogen atom, hydroxyl, nitro, cyano, amino, carboxyl, a C₂₋₈ acyl group, or an alkoxycarbonyl group comprising a C₁₋₈ alkoxy moiety; and

each of R⁴ and R⁵ independently is hydrogen, a C₁₋₈ alkyl group, or a C₁₋₈ alkyl group having one to three halogen atoms.

The invention further relates to an agent for preventing or treating neuropathic pain associated with Guillain-Barré syndrome containing a compound having the following formula (IX) or a pharmacologically acceptable salt thereof as an active ingredient:

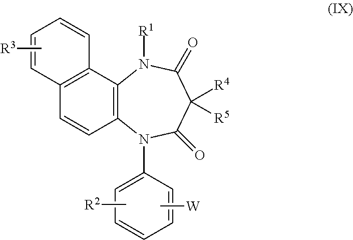

wherein R¹ is hydrogen, a C₁₋₈ alkyl group, a C₂₋₈ alkenyl group, a C₁₋₈ alkyl group having one to three halogen atoms, or a C₁₋₃ alkyl group having phenyl;

each of R² and R³ independently is hydrogen, a C₁₋₈ alkyl group, a C₁₋₈ alkoxy group, a C₁₋₈ alkyl group having one to three halogen atoms, a C₁₋₈ alkoxy group having one to three halogen atoms, a halogen atom, hydroxyl, nitro, cyano, amino, a C₁₋₈ alkylamino group, a C₂₋₈ dialkylamino group, a C₂₋₈ acylamino group, a C₂₋₈ acylamino group having one to three halogen atoms, a C₁₋₈ alkylsulfonylamino group, carboxyl, a C₂₋₈ acyl group, an alkoxycarbonyl group comprising a C₁₋₈ alkoxy moiety, carbamoyl, a C₁₋₈ alkylthio group, a alkylsulfinyl group, a C₁₋₈ alkylsulfonyl group, or sulfamoyl;

each of R⁴ and R⁵ independently is hydrogen, a C₁₋₈ alkyl group, a alkyl group having one to three halogen atoms, or a C₁₋₈ alkyl group having phenyl; and

W is a five-membered or six-membered heterocyclic ring optionally having one or more substituents and comprising one to four nitrogen atoms as the members of the ring.

The invention further relates to an agent for preventing or treating neuropathic pain associated with Guillain-Barré syndrome containing 5-[3-(1H-tetrazol-5-yl)phenyl]-1H-naphtho[1,2-b][1,4]diazepine-2,4(3H,5H)-dione potassium salt as an active ingredient.

The invention further relates to an agent for preventing or treating neuropathic pain associated with Guillain-Barré syndrome containing paroxetine or a pharmacologically acceptable salt thereof as an active ingredient.

### BRIEF DESCRIPTIONS OF THE DRAWINGS

FIG. 1 shows immunostaining images of Iba1-positive cell signals (upper figures) or P2X₄ receptor-positive signals (lower figures). The left figures show controls, and the right figures show EAN models.

FIG. 2 shows influence of preventive administration of the compound A on pain threshold of EAN rat model.

FIG. 3 shows influence of therapeutic administration of the compound A on pain threshold of EAN rat model.

### THE EMBODIMENTS OF THE INVENTION

The present invention is described below in more detail.

The active ingredients of the agent of the invention for preventing or treating neuropathic pain associated with Guillain-Barré syndrome include the following compounds.
(1) P2X₄ receptor antagonist.
(2) A compound having the following formula or a pharmacologically acceptable salt thereof:

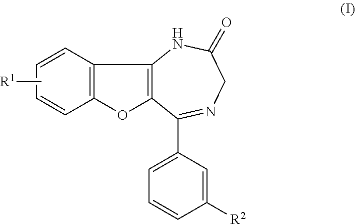

wherein R¹ is a halogen atom; and

R² is hydrogen, a halogen atom, nitro, cyano, —C(O)—OR³, —C(O)—NR⁴R⁵, —SO₂—OR³, or —SO₂—NR⁴R⁵, wherein each of R³, R⁴, and R⁵ is hydrogen or a C₁₋₆ alkyl group; or in the alternative

R¹ is hydrogen; and

R² is a halogen atom, nitro, cyano, —C(O)—OR³, —C(O)—NR⁴R⁵, —SO₂—OR³, or —SO₂—NR⁴R⁵, wherein each of R³, R⁴, and R⁵ is hydrogen or a C₁₋₆ alkyl group.
(3) A compound having the formula (I) described in (2) or a pharmacologically acceptable salt thereof:
   wherein R^{T} is chloro or bromo; and

R² is hydrogen, chloro, bromo, nitro, cyano, —C(O)—OR³, or —C(O)—NR⁴R⁵, wherein each of R³, R⁴, and R⁵ is hydrogen or a alkyl group; or in the alternative

R¹ is hydrogen; and

R² is chloro, bromo, nitro, cyano, —C(O)—OR³, or —C(O)—NR⁴R⁵, wherein each of R³, R⁴, and R⁵ is hydrogen or a C₁₋₄ alkyl group.
(4) A compound having the following formula (Ia) or a pharmacologically acceptable salt thereof:

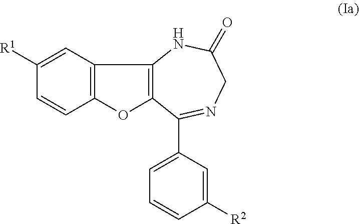

wherein R^{l} is chloro or bromo; and

R² is hydrogen, chloro, bromo, nitro, or cyano; or in the alternative

R¹ is hydrogen; and

R² is chloro, bromo, nitro, or cyano.
(5) A compound having the following formula (II) or a pharmacologically acceptable salt thereof:

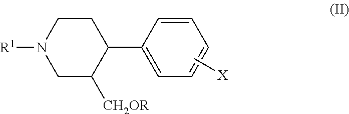

wherein R is a C₁₋₄ alkyl group, a C₂₋₄ alkynyl group, phenyl (optionally having one or more substituents selected from the group consisting of a lower alkyl group, an alkylthio group, an alkoxy group, a halogen atom, nitro, an acylamino group, methylsulfonyl, and methylenedioxy), or tetrahydronaphthyl;

R¹ is hydrogen; and

X is hydrogen, a C₁₋₄ alkyl group, a trifluoroalkyl group, hydroxyl, a halogen atom, methylthio, or an arylalkoxy group.
(6) A selective serotonin reuptake inhibitor.
(7) Imipramine, nortriptyline, amitriptyline, desipramine, doxepin, fluoxetine, fluvoxamine, citalopram, or a pharmacologically acceptable salt thereof.
(8) A compound having the following formula (III) or a pharmacologically acceptable salt thereof:

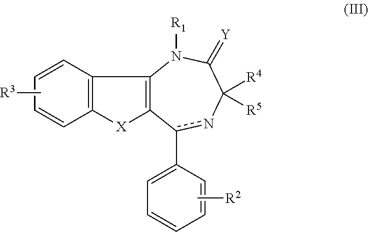

wherein X is S or CH₂;

Y is O, S, or NH;

R¹ is hydrogen, a C₁₋₈ alkyl group, a C₁₋₈ alkyl group having one or more halogen atoms, an aralkyl group comprising a C₁₋₆ alkyl moiety and a C₆₋₁₀ aryl moiety, a C₂₋₈ alkenyl group, carboxymethyl, or an alkoxycarbonylmethyl group comprising a C₁₋₈ alkoxy moiety;

each of R² and R³ independently is hydrogen, a C₁₋₈ alkyl group, a C₁₋₈ alkoxy group, a C₁₋₉ alkyl group having one or more halogen atoms, a C₁₋₈ alkoxy group having one or more halogen atoms, a halogen atom, amino, carboxyl, hydroxyl, nitro, cyano, a C₂₋₈ acyl group, a C₆₋₁₀ aryl group, or a five-membered or six-membered heterocyclic group;

each of R⁴ and R⁵ independently is hydrogen, a C₁₋₈ alkyl group, or a C₁₋₈ alkyl group having one or more halogen atoms; and

the double line consisting of a broken line and a solid line is a single bond or a double bond.
(9) A compound having the formula (III) described in (8) or a pharmacologically acceptable salt thereof, wherein X is S.
(10) A compound having the formula (III) described in (8) or a pharmacologically acceptable salt thereof, wherein Y is O.
(11) A compound having the formula (III) described in (8) or a pharmacologically acceptable salt thereof, wherein R¹ is hydrogen or a C₁₋₈ alkyl group.
(12) A compound having the formula (III) described in (8) or a pharmacologically acceptable salt thereof, wherein each of R² and R³ independently is hydrogen, a C₁₋₈ alkyl group, a C₁₋₈ alkoxy group, a C₁₋₈ alkyl group having one or more halogen atoms, a C₁₋₈ alkoxy group having one or more halogen atoms, a halogen atom, amino, carboxyl, hydroxyl, nitro, or cyano.
(13) A compound having the formula (III) described in (8) or a pharmacologically acceptable salt thereof, wherein R³ is hydrogen, and R² is a halogen atom or hydroxyl.
(14) A compound having the formula (III) described in (8) or a pharmacologically acceptable salt thereof, wherein R² substitutes at meta-position.
(15) A compound having the formula (III) described in (8) or a pharmacologically acceptable salt thereof, wherein each of R⁴ and R⁵ is hydrogen.
(16) A compound having the formula (III) described in (8) or a pharmacologically acceptable salt thereof, wherein the double line consisting of a broken line and a solid line is a double bond.
(17) A compound having the following formula (IV) or a pharmacologically acceptable salt thereof:

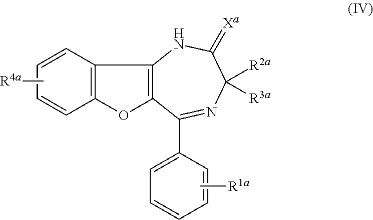

wherein X^{a} is O, S, or NH;

R^{1a} is hydroxyl, tetrazolyl, N(R^{5a})(R^{6a}), a C₂₋₈ alkenyl group, a C₂₋₈ alkynyl group, a C₁₋₈ alkyl group having one or more halogen atoms, a C₁₋₈ alkoxy group having one or more halogen atoms, or a C₆₋₁₀ aryl group, wherein R^{5a} is hydrogen or a C₁₋₈ alkyl group, and R^{6a} is hydrogen, a C₁₋₈ alkyl group, or a C₂₋₈ acyl group;

each of R^{2a} and R^{3a} independently is hydrogen, a C₁₋₈ alkyl group, or a C₁₋₈ alkyl group having one or more halogen atoms; and

R^{4a} is hydrogen, a C₁₋₈ alkyl group, a C₁₋₈ alkoxy group, a C₁₋₈ alkyl group having one or more halogen atoms, a halogen atom, hydroxyl, nitro, amino, carboxyl, tetrazolyl, cyano, a C₆₋₁₀ aryl group, or a five-membered or six-membered heterocyclic group.
(18) A compound having the formula (IV) described in (17) or a pharmacologically acceptable salt thereof, wherein X^{a} is O.
(19) A compound having the formula (IV) described in (17) or a pharmacologically acceptable salt thereof, wherein R^{1a} is hydroxyl, amino, a C₁₋₈ alkylamino group, a C₂₋₁₂ dialkylamino group, a C₁₋₈ alkyl group having one or more halogen atoms, or phenyl.
(20) A compound having the formula (IV) described in (17) or a pharmacologically acceptable salt thereof, wherein R^{1a} substitutes at meta-position.
(21) A compound having the formula (IV) described in (17) or a pharmacologically acceptable salt thereof, wherein each of R^{2a} and R^{3a} is hydrogen.
(22) A compound having the formula (IV) described in (17) or a pharmacologically acceptable salt thereof, wherein R^{4a} is hydrogen.
(23) A compound having the following formula (IVa) or a pharmacologically acceptable salt thereof:

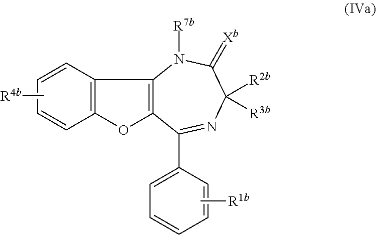

wherein X^{b} is O, S, or NH;

R^{1b} is a halogen atom, hydroxyl, tetrazolyl, N(R^{5b})(R^{6b}), a C₂₋₈ alkenyl group, a C₂₋₈ alkynyl group, a C₁₋₈ alkyl group having one or more halogen atoms, a C₁₋₈ alkoxy group having one or more halogen atoms, or a C₆₋₁₀ aryl group, wherein R^{5b} is hydrogen or a C₁₋₈ alkyl group, and R^{6b} is hydrogen, a C₁₋₈ alkyl group, or a C₂₋₈ acyl group;

each of R^{2b} and R^{3b} independently is hydrogen, a C₁₋₈ alkyl group, or a C₁₋₈ alkyl group having one or more halogen atoms;

R^{4b} is hydrogen, a C₁₋₈ alkyl group, an alkoxy group, a C₁₋₈ alkyl group having one or more halogen atoms, a halogen atom, hydroxyl, nitro, amino, carboxyl, tetrazolyl, cyano, a C₆₋₁₀ aryl group, or a five-membered or six-membered heterocyclic group; and

R^{7b} is a alkyl group.
(24) A compound having the formula (IVa) described in (23) or a pharmacologically acceptable salt thereof, wherein X^{b} is O.
(25) A compound having the formula (IVa) described in (23) or a pharmacologically acceptable salt thereof, wherein R^{1b} is a halogen atom, hydroxyl, amino, a C₁₋₈ alkylamino group, a dialkylamino group, a C₁₋₈ alkyl group having one or more halogen atoms, or phenyl.
(26) A compound having the formula (IVa) described in (23) or a pharmacologically acceptable salt thereof, wherein R^{1b} substitutes at meta-position.
(27) A compound having the formula (IVa) described in (23) or a pharmacologically acceptable salt thereof, wherein each of R^{2b} and R^{3b} is hydrogen.
(28) A compound having the formula (IVa) described in (23) or a pharmacologically acceptable salt thereof, wherein R^{4b} is hydrogen.
(29) A compound having the following formula (IVb) or a pharmacologically acceptable salt thereof:

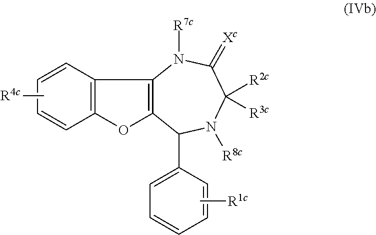

wherein X^{c} is O, S, or NH;

R^{1c} is hydrogen, a halogen atom, a C₁₋₈ alkyl group, a C₁₋₈ alkoxy group, hydroxyl, tetrazolyl, N(R^{5c})(R^{6c}), a C₂₋₈ alkenyl group, a C₂₋₈ alkynyl group, a C₁₋₈ alkyl group having one or more halogen atoms, a C₁₋₈ alkoxy group having one or more halogen atoms, or a C₆₋₁₀ aryl group, wherein R^{5c} is hydrogen or a C₁₋₈ alkyl group, and R^{6c} is hydrogen, a C₁₋₈ alkyl group, or a C₂₋₈ acyl group;

each of R^{2c} and R^{3c} independently is hydrogen, a C₁₋₈ alkyl group, or a C₁₋₈ alkyl group having one or more halogen atoms;

R^{4c} is hydrogen, a C₁₋₈ alkyl group, an alkoxy group, a C₁₋₈ alkyl group having one or more halogen atoms, a halogen atom, hydroxyl, nitro, amino, carboxyl, tetrazolyl, cyano, a C₆₋₁₀ aryl group, or a five-membered or six-membered heterocyclic group;

R^{7c} is hydrogen or a C₁₋₈ alkyl group; and

R^{8c} is hydrogen, a C₁₋₈ alkyl group, or a C₂₋₈ acyl group.
(30) A compound having the formula (IVb) described in (29) or a pharmacologically acceptable salt thereof, wherein X′ is O.
(31) A compound having the formula (IVb) described in (29) or a pharmacologically acceptable salt thereof, wherein R^{1c} is hydrogen, a halogen atom, hydroxyl, amino, a alkylamino group, a dialkylamino group, a C₁₋₈ alkyl group having one or more halogen atoms, or phenyl.
(32) A compound having the formula (IVb) described in (29) or a pharmacologically acceptable salt thereof, wherein R substitutes at meta-position.
(33) A compound having the formula (IVb) described in (29) or a pharmacologically acceptable salt thereof, wherein each of R^{2c} and R^{3c} is hydrogen.
(34) A compound having the formula (IVb) described in (29) or a pharmacologically acceptable salt thereof, wherein R^{4c} is hydrogen or a halogen atom.
(35) A compound having the formula (IVb) described in (29) or a pharmacologically acceptable salt thereof, wherein R is hydrogen.
(36) A compound having the formula (IVb) described in (29) or a pharmacologically acceptable salt thereof, wherein R^{8c} is hydrogen.
(37) A compound having the following formula (V) or a pharmacologically acceptable salt thereof:

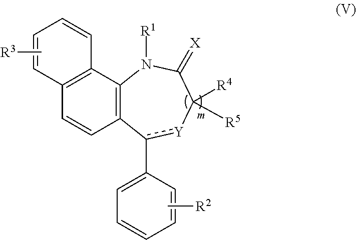

wherein X is O, S, or NH;

Y is N or NR⁶, wherein R⁶ is hydrogen or a C₁₋₈ alkyl group;

R¹ is hydrogen, a C₁₋₈ alkyl group, a C₂₋₈ alkenyl group, a C₁₋₈ alkyl group having one to three halogen atoms, or an alkyl group having phenyl;

R² is a C₁₋₈ alkyl group, a C₁₋₈ alkoxy group, a C₁₋₈ alkyl group having one to three halogen atoms, hydroxyl, nitro, amino, carboxyl, tetrazolyl, or cyano;

R³ is hydrogen, a C₁₋₈ alkyl group, a C₁₋₈ alkoxy group, a C₁₋₈ alkyl group having one to three halogen atoms, a halogen atom, hydroxyl, nitro, amino, carboxyl, tetrazolyl, or cyano;

each of R⁴ and R⁵ independently is hydrogen, a C₁₋₈ alkyl group, or a C₁₋₈ alkyl group having one to three halogen atoms;

m is 1 or 2;

when Y is N, the double line consisting of a solid line and a broken line is a double bond; and

when Y is NR⁶, the double line consisting of a solid line and a broken line is a single bond.
(38) A compound having the formula (V) described in (37) or a pharmacologically acceptable salt thereof, wherein m is 1.
(39) A compound having the formula (V) described in (37) or a pharmacologically acceptable salt thereof, wherein X is O.
(40) A compound having the formula (V) described in (37) or a pharmacologically acceptable salt thereof, wherein Y is N.
(41) A compound having the formula (V) described in (37) or a pharmacologically acceptable salt thereof, wherein R¹ is hydrogen or a C₁₋₆ alkyl group.
(42) A compound having the formula (V) described in (37) or a pharmacologically acceptable salt thereof, wherein R¹ is hydrogen.
(43) A compound having the formula (V) described in (37) or a pharmacologically acceptable salt thereof, wherein each of R⁴ and R⁵ is hydrogen.
(44) A compound having the formula (V) described in (37) or a pharmacologically acceptable salt thereof, wherein R² is a C₁₋₈ alkyl group, a C₁₋₈ alkoxy group, a C₁₋₈ alkyl group having one to three halogen atoms, or hydroxyl.
(45) A compound having the formula (V) described in (37) or a pharmacologically acceptable salt thereof, wherein R² is a C₁₋₈ alkoxy group or hydroxyl.
(46) A compound having the formula (V) described in (37) or a pharmacologically acceptable salt thereof, wherein R³ is hydrogen or a halogen atom.
(47) A compound having the formula (V) described in (37) or a pharmacologically acceptable salt thereof, wherein R³ is hydrogen.
(48) A compound having the following formula (Va) or a pharmacologically acceptable salt thereof:

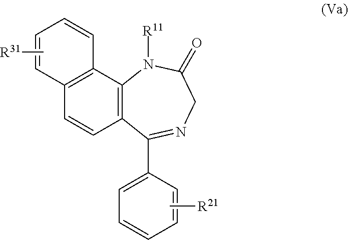

wherein R¹¹ is hydrogen or a C₁₋₈ alkyl group;

R²¹ is a C₁₋₈ alkyl group, a C₁₋₈ alkoxy group, a C₁₋₈ alkyl group having one to three halogen atoms, or hydroxyl; and

R³¹ is hydrogen or a halogen atom.
(49) A compound having the formula (Va) described in (48) or a pharmacologically acceptable salt thereof, wherein R¹¹ is hydrogen.
(50) A compound having the formula (Va) described in (48) or a pharmacologically acceptable salt thereof, wherein R²¹ is a C₁₋₈ alkoxy group or hydroxyl.
(51) A compound having the formula (Va) described in (48) or a pharmacologically acceptable salt thereof, wherein R³¹ is hydrogen.
(52) 5-(3-methoxyphenyl)-1,3-dihydro-2H-naphtho[1,2-e]-1,4-diazepin-2-one,

5-(3-hydroxyphenyl)-1,3-dihydro-2H-naphtho[1,2-e]-1,4-diazepin-2-one,

5-(4-methoxyphenyl)-1,3-dihydro-2H-naphtho[1,2-e]-1,4-diazepin-2-one,

5-(4-hydroxyphenyl)-1,3-dihydro-2H-naphtho[1,2-e]-1,4-diazepin-2-one,

5-(4-methylphenyl)-1,3-dihydro-2H-naphtho[1,2-e]-1,4-diazepin-2-one,

5-(2-methoxyphenyl)-1,3-dihydro-2H-naphtho[1,2-e]-1,4-diazepin-2-one,

5-(2-hydroxyphenyl)-1,3-dihydro-2H-naphtho[1,2-e]-1,4-diazepin-2-one,

5-(3,4-dimethoxyphenyl)-1,3-dihydro-2H-naphtho[1,2-e]-1,4-diazepin-2-one,

5-(3,4-dihydroxyphenyl)-1,3-dihydro-2H-naphtho[1,2-e]-1,4-diazepin-2-one, or

a pharmacologically acceptable salt thereof.
(53) A compound having the following formula (VI) or a pharmacologically acceptable salt thereof:

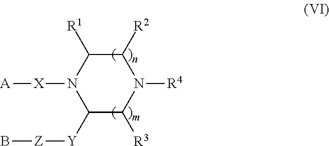

wherein A is an aryl group optionally having one or more substituents or a heterocyclic group optionally having one or more substituents;

B is an aryl group optionally having one or more substituents or a heterocyclic group optionally having one or more substituents;

X is a C₁₋₅ alkylene group or a bond;

Y is a C₁₋₅ alkylene group optionally comprising a double bond;

Z is O, S, N(R⁵), or a bond, wherein R is hydrogen or a C₁₋₈ alkyl group;

each of R¹, R², and R³ independently is hydrogen, a C₁₋₈ alkyl group, or a C₁₋₈ alkyl group having one to three halogen atoms;

R⁴ is hydrogen, a C₁₋₈ alkyl group, a C₁₋₈ alkyl group having one to three halogen atoms, a three-membered to seven-membered cycloalkyl group, or a C₁₋₈ alkyl group having a three-membered to seven-membered cycloalkyl group; and

each of n and m independently is 1 or 2;

provided that when X is a bond, the substituent of the aryl group represented by A is not an alkyl group.
(54) A compound having the formula (VI) described in (53) or a pharmacologically acceptable salt thereof, wherein A is phenyl or thienyl, each of which optionally has one to three substituents selected from the group consisting of a halogen atom, a C₁₋₈ alkyl group (except that X is a bond), a C₁₋₈ alkyl group having one to three halogen atoms, nitro, cyano, hydroxyl, amino, a C₁₋₈ alkylamino group, a C₂₋₁₆ dialkylamino group, a C₂₋₈ acylamino group, a C₁₋₈ alkoxy group, a C₁₋₈ alkoxy group having one to three halogen atoms, an aryl group, and a heterocyclic group.
(55) A compound having the formula (VI) described in (53) or a pharmacologically acceptable salt thereof, wherein A is phenyl optionally having one to three substituents selected from the group consisting of a halogen atom, a C₁₋₈ alkyl group (except that X is a bond), a C₁₋₈ alkoxy group, and a C₁₋₈ alkyl group having one to three halogen atoms.
(56) A compound having the formula (VI) described in (53) or a pharmacologically acceptable salt thereof, wherein B is phenyl, naphthyl, benzofuranyl, 1,3-benzo[d]dioxolyl, quinolyl, indolyl, benzothienyl, thienyl, or pyridyl, each of which optionally has one to three substituents selected from the group consisting of a halogen atom, a C₁₋₈ alkyl group, a C₁₋₈ alkyl group having one to three halogen atoms, nitro, cyano, hydroxyl, amino, a C₁₋₈ alkylamino group, a C₂₋₁₆ dialkylamino group, a C₂₋₈ acylamino group, a C₁₋₈ alkoxy group, a C₁₋₈ alkoxy group having one to three halogen atoms, a C₆₋₁₂ aryloxy group, a C₂₋₉ alkoxycarbonyl group, carbamoyl, a C₂₋₉ alkylcarbamoyl group, sulfamoyl, a C₁₋₈ alkylsulfamoyl group, and a C₂₋₁₆ dialkylsulfamoyl group.
(57) A compound having the formula (VI) described in (53) or a pharmacologically acceptable salt thereof, wherein B is phenyl, naphthyl, benzofuranyl, or 1,3-benzo[d]dioxolyl, each of which optionally has one to three substituents selected from the group consisting of a halogen atom, a C₁₋₈ alkyl group, a C₁₋₈ alkyl group having one to three halogen atoms, a C₁₋₈ alkoxy group, a C₆₋₁₂ aryloxy group, sulfamoyl, a C₁₋₈ alkylsulfamoyl group, and a C₂₋₁₆ dialkylsulfamoyl group.
(58) A compound having the formula (VI) described in (53) or a pharmacologically acceptable salt thereof, wherein X is a bond.
(59) A compound having the formula (VI) described in (53) or a pharmacologically acceptable salt thereof, wherein Y is methylene.
(60) A compound having the formula (VI) described in (53) or a pharmacologically acceptable salt thereof, wherein Z is O or S.
(61) A compound having the formula (VI) described in (53) or a pharmacologically acceptable salt thereof, wherein each of R¹, R², and R³ is hydrogen.
(62) A compound having the formula (VI) described in (53) or a pharmacologically acceptable salt thereof, wherein R⁴ is hydrogen or a C₁₋₈ alkyl group.
(63) A compound having the formula (VI) described in (53) or a pharmacologically acceptable salt thereof, wherein R⁴ is hydrogen.
(64) A compound having the formula (VI) described in (53) or a pharmacologically acceptable salt thereof, wherein each of n and m is 1.
(65) A compound having the following formula (VIa) or a pharmacologically acceptable salt thereof:

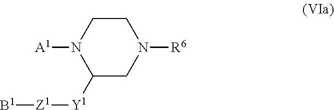

wherein A¹ is phenyl or thienyl, each of which optionally has one to three substituents selected from the group consisting of a halogen atom, a C₁₋₈ alkyl group having one to three halogen atoms, nitro, cyano, hydroxyl, amino, a alkylamino group, a C₂₋₁₆ dialkylamino group, a C₂₋₈ acylamino group, a C₁₋₈ alkoxy group, a C₁₋₈ alkoxy group having one to three halogen atoms, an aryl group, and a heterocyclic group;

B¹ is an aryl group optionally having one or more substituents or a heterocyclic group optionally having one or more substituents;

Y¹ is a C₁₋₆ alkylene chain optionally comprising a double bond;

Z¹ is O, S, N(R⁷), or a bond, wherein R⁷ is hydrogen or a C₁₋₈ alkyl group; and

R⁶ is hydrogen, a C₁₋₈ alkyl group, a alkyl group having one to three halogen atoms, or a three-membered to seven-membered cycloalkyl group.
(66) A compound having the formula (VIa) described in (65) or a pharmacologically acceptable salt thereof, wherein A¹ is phenyl optionally having one to three substituents selected from the group consisting of a halogen atom, a C₁₋₈ alkyl group having one to three halogen atoms, and a C₁₋₈ alkoxy group.
(67) A compound having the formula (VIa) described in (65) or a pharmacologically acceptable salt thereof, wherein B is phenyl, naphthyl, benzofuranyl, 1,3-benzo[d]dioxolyl, quinolyl, indolyl, benzothienyl, thienyl, or pyridyl, each of which optionally has one to three substituents selected from the group consisting of a halogen atom, a C₁₋₈ alkyl group, a C₁₋₈ alkyl group having one to three halogen atoms, nitro, cyano, hydroxyl, amino, a C₁₋₈ alkylamino group, a C₂₋₁₆ dialkylamino group, a C₂₋₈ acylamino group, a C₁₋₈ alkoxy group, a C₁₋₈ alkoxy group having one to three halogen atoms, a C₆₋₁₂ aryloxy group, a C₂₋₉ alkoxycarbonyl group, carbamoyl, a C₂₋₉ alkylcarbamoyl group, sulfamoyl, a C₁₋₈ alkylsulfamoyl group, and a C₂₋₁₆ dialkylsulfamoyl group.
(68) A compound having the formula (VIa) described in (65) or a pharmacologically acceptable salt thereof, wherein B¹ is phenyl, naphthyl, benzofuranyl, or 1,3-benzo[d]dioxolyl, each of which optionally has one to three substituents selected from the group consisting of a halogen atom, a C₁₋₈ alkyl group, a C₁₋₆ alkyl group having one to three halogen atoms, a C₁₋₈ alkoxy group, a C₆₋₁₂ aryloxy group, sulfamoyl, a C₁₋₈ alkylsulfamoyl group, and a C₂₋₁₆ dialkylsulfamoyl group.
(69) A compound having the formula (VIa) described in (65) or a pharmacologically acceptable salt thereof, wherein Y¹ is methylene.
(70) A compound having the formula (VIa) described in (65) or a pharmacologically acceptable salt thereof, wherein Z¹ is O or S.
(71) A compound having the formula (VIa) described in (65) or a pharmacologically acceptable salt thereof, wherein R⁶ is hydrogen or a C₁₋₈ alkyl group.
(72) A compound having the formula (VIa) described in (65) or a pharmacologically acceptable salt thereof, wherein R⁶ is hydrogen.
(73) A compound having the following formula (VIb) or a pharmacologically acceptable salt thereof:

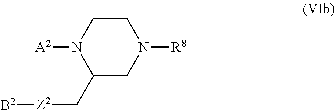

wherein A² is phenyl or thienyl, each of which optionally has one to three substituents selected from the group consisting of a halogen atom, a C₁₋₈ alkyl group having one to three halogen atoms, nitro, cyano, acetylamino, a C₁₋₈ alkoxy group, a C₁₋₈ alkoxy group having one to three halogen atoms, an aryl group, and a heterocyclic group;

B² is phenyl, naphthyl, benzofuranyl, 1,3-benzo[d]dioxolyl, guinolyl, indolyl, benzothienyl, thienyl, or pyridyl, each of which optionally has one to three substituents selected from the group consisting of a halogen atom, a C₁₋₈ alkyl group, a C₁₋₈ alkyl group having one to three halogen atoms, nitro, cyano, hydroxyl, amino, a C₂₋₈ acylamino group, a C₁₋₈ alkoxy group, a C₁₋₈ alkoxy group having one to three halogen atoms, a C₆₋₁₂ aryloxy group, sulfamoyl, a C₁₋₈ alkylsulfamoyl group, and a C₂₋₁₆ dialkylsulfamoyl group;

Z² is O, S, or NH; and

R⁸ is hydrogen or a C₁₋₈ alkyl group.
(74) A compound having the formula (VIb) described in (73) or a pharmacologically acceptable salt thereof, wherein A² is phenyl optionally having one to three substituents selected from the group consisting of a halogen atom, a C₁₋₈ alkyl group having one to three halogen atoms, a C₁₋₈ alkoxy group, nitro, cyano, or acetylamino.
(75) A compound having the formula (VIb) described in (73) or a pharmacologically acceptable salt thereof, wherein A² is phenyl optionally having one to three substituents selected from the group consisting of a halogen atom, a C₁₋₈ alkyl group having one to three halogen atoms, and a C₁₋₈ alkoxy group.
(76) A compound having the formula (VIb) described in (73) or a pharmacologically acceptable salt thereof, wherein B² is phenyl, naphthyl, benzofuranyl, or 1,3-benzo[d]dioxolyl, each of which optionally has one to three substituents selected from the group consisting of a halogen atom, a C₁₋₈ alkyl group, a C₁₋₈ alkyl group having one to three halogen atoms, an aryloxy group, sulfamoyl, a C₁₋₈ alkylsulfamoyl group, and a C₂₋₁₆ dialkylsulfamoyl group.
(77) A compound having the formula (VIb) described in (73) or a pharmacologically acceptable salt thereof, wherein Z² is O or S.
(78) A compound having the formula (VIb) described in (73) or a pharmacologically acceptable salt thereof, wherein R is hydrogen.
(79) 1-(4-fluorophenyl)-2-(4-phenoxyphenoxymethyl)piperazine,

1-(4-fluorophenyl)-2-(4-phenoxyphenylsulfanylmethyl)piperazine,

2-(4-chlorophenoxymethyl)-1-(4-isopropoxyphenyl)piperazine,

2-(2,4-dichlorophenoxymethyl)-1-(4-isopropoxyphenyl)piperazine,

2-(4-tert-butoxyphenoxymethyl)-1-(4-isopropoxyphenyl)piperazine,

2-(4-chlorophenoxymethyl)-1-(3-methoxyphenyl)piperazine,

2-(4-chlorophenoxymethyl)-1-(2-methoxyphenyl)piperazine, or

a pharmacologically acceptable salt thereof.
(80) A compound having the following formula (VII) or a pharmacologically acceptable salt thereof:

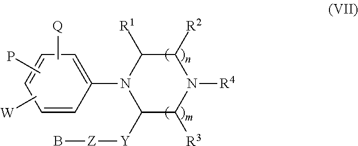

wherein B is an aryl group optionally having one or more substituents or a heterocyclic group optionally having one or more substituents;

Y is a C₁₋₅ alkylene group optionally comprising a double bond;

Z is O, S, N(R⁵), or a bond, wherein R⁵ is hydrogen or a C₁₋₈ alkyl group;

each of R¹, R², and R³ independently is hydrogen, a C₁₋₈ alkyl group, or a C₁₋₈ alkyl group having one to three halogen atoms;

R⁴ is hydrogen, a C₁₋₈ alkyl group, a C₁₋₈ alkyl group having one to three halogen atoms, a three-membered to seven-membered cycloalkyl group, or a C₁₋₈ alkyl group having a three-membered to seven-membered cycloalkyl group;

each of P and Q independently is hydrogen, a halogen atom, a C₁₋₈ alkyl group, a C₁₋₈ alkyl group having one to three halogen atoms, nitro, cyano, hydroxyl, amino, a C₁₋₈ alkylamino group, a C₂₋₁₆ dialkylamino group, a C₂₋₈ acylamino group, a C₁₋₈ alkoxy group, a C₁₋₈ alkoxy group having one to three halogen atoms, or a heterocyclic group;

W is a C₁₋₈ alkyl group or a three-membered to seven-membered cycloalkyl group; or

when P and W are placed at 2- and 3-positions or 3- and 4-positions of phenyl, P and W are combined to form propylene or tetramethylene; and

each of n and m independently is 1 or 2.
(81) A compound having the formula (VII) described in (80) or a pharmacologically acceptable salt thereof, wherein B is phenyl, naphthyl, benzofuranyl, indolyl, benzothienyl, or thienyl optionally having one to three substituents selected from the group consisting of a halogen atom, a C₁₋₈ alkyl group, a C₁₋₈ alkyl group having one to three halogen atoms, nitro, cyano, hydroxyl, amino, a C₁₋₈ alkylamino group, a C₂₋₁₆ dialkylamino group, a C₂₋₈ acylamino group, a C₁₋₈ alkoxy group, a alkoxy group having one to three halogen atoms, a C₆₋₁₂ aryloxy group, an arylalkoxy group comprising a C₁₋₈ alkyl moiety, a C₂₋₉ alkoxycarbonyl group, carbamoyl, a C₂₋₉ alkylcarbamoyl group, sulfamoyl, a C₁₋₈ alkylsulfamoyl group, and a C₂₋₁₆ dialkylsulfamoyl group.
(82) A compound having the formula (VII) described in (80) or a pharmacologically acceptable salt thereof, wherein B is phenyl optionally having one to three substituents selected from the group consisting of a halogen atom, a C₁₋₈ alkyl group, a C₁₋₈ alkyl group having one to three halogen atoms, nitro, cyano, hydroxyl, amino, a C₁₋₈ alkylamino group, a C₂₋₁₆ dialkylamino group, a C₂₋₈ acylamino group, a C₁₋₈ alkoxy group, a C₁₋₈ alkoxy group having one to three halogen atoms, a C₆₋₁₂ aryloxy group, an arylalkoxy group comprising a C₁₋₈ alkyl moiety, a C₂₋₉ alkoxycarbonyl group, carbamoyl, a C₂₋₉ alkylcarbamoyl group, sulfamoyl, a C₁₋₈ alkylsulfamoyl group, and a C₂₋₁₆ dialkylsulfamoyl group.
(83) A compound having the formula (VII) described in (80) or a pharmacologically acceptable salt thereof, wherein each of P and Q independently is hydrogen, a C₁₋₈ alkyl group, a C₁₋₈ alkyl group having one to three halogen atoms, or a C₁₋₈ alkoxy group.
(84) A compound having the formula (VII) described in (80) or a pharmacologically acceptable salt thereof, wherein each of P and Q is hydrogen.
(85) A compound having the formula (VII) described in (80) or a pharmacologically acceptable salt thereof, wherein W is a C₃₋₆ alkyl group.
(86) A compound having the formula (VII) described in (80) or a pharmacologically acceptable salt thereof, wherein W is n-propyl, isopropyl, n-butyl, or isobutyl.
(87) A compound having the formula (VII) described in (80) or a pharmacologically acceptable salt thereof, wherein each of n and m is 1.
(88) A compound having the formula (VII) described in (80) or a pharmacologically acceptable salt thereof, wherein Y is methylene.
(89) A compound having the formula (VII) described in (80) or a pharmacologically acceptable salt thereof, wherein Z is O or S.
(90) A compound having the formula (VII) described in (80) or a pharmacologically acceptable salt thereof, wherein each of R¹, R², and R³ is hydrogen.
(91) A compound having the formula (VII) described in (80) or a pharmacologically acceptable salt thereof, wherein R⁴ is hydrogen or a C₁₋₈ alkyl group.
(92) A compound having the formula (VII) described in (80) or a pharmacologically acceptable salt thereof, wherein R⁴ is hydrogen.
(93) A compound having the formula (VII) described in (80) or a pharmacologically acceptable salt thereof: wherein R⁴ is hydrogen;

Y is methylene;

Z is O or S; and

B is phenyl optionally having one to three substituents selected from the group consisting of a halogen atom, a C₁₋₈ alkyl group, a C₁₋₈ alkyl group having one to three halogen atoms, cyano, hydroxyl, a C₁₋₈ alkoxy group, a C₁₋₈ alkoxy group having one to three halogen atoms, benzyloxy, sulfamoyl, and a C₁₋₈ alkylsulfamoyl group.
(94) 2-(4-chlorophenoxymethyl)-1-(4-isopropylphenyl)piperazine,

2-(4-chlorophenoxymethyl)-1-(4-propylphenyl)piperazine,

2-(4-chlorophenoxymethyl)-1-(3-isopropylphenyl)piperazine,

2-(4-chlorophenoxymethyl)-1-(2,4,6-trimethylphenyl)piperazine,

2-(4-chlorophenoxymethyl)-1-indan-5-yl-piperazine,

1-(4-isopropylphenyl)-2-[4-(isopropylsulfamoyl)phenoxymethyl]piperazine,

2-(4-chlorophenylsulfanylmethyl)-1-(4-isopropylphenyl)piperazine,

1-(3-isopropylphenyl)-2-[4-(isopropylsulfamoyl)phenoxymethyl]piperazine,

1-(4-isopropylphenyl)-2-(4-phenoxyphenoxymethyl)piperazine, or

a pharmacologically acceptable salt thereof.
(95) A compound having the following formula (VIII) or a pharmacologically acceptable salt thereof:

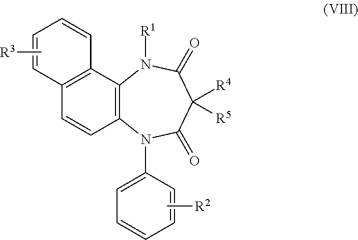

wherein R¹ is hydrogen, a C₁₋₈ alkyl group, a C₂₋₈ alkenyl group, a C₁₋₈ alkyl group having one to three halogen atoms, or a C₁₋₃ alkyl group having phenyl;

R² is hydrogen, a C₁₋₈ alkyl group, a C₁₋₈ alkoxy group, a C₁₋₈ alkyl group having one to three halogen atoms, a C₁₋₈ alkoxy group having one to three halogen atoms, a halogen atom, hydroxyl, nitro, cyano, amino, a C₁₋₈ alkylamino group, a C₂₋₈ dialkylamino group, a C₂₋₈ acylamino group, a C₂₋₈ acylamino group having one to three halogen atoms, a C₁₋₈ alkylsulfonylamino group, carboxyl, a C₂₋₈ acyl group, an alkoxycarbonyl group comprising a C₁₋₈ alkoxy moiety, carbamoyl, a C₁₋₈ alkylthio group, a C₁₋₈ alkylsulfinyl group, a C₁₋₈ alkylsulfonyl group, or sulfamoyl;

R³ is hydrogen, a C₁₋₈ alkyl group, a C₁₋₈ alkoxy group, a C₁₋₈ alkyl group having one to three halogen atoms, a C₁₋₈ alkoxy group having one to three halogen atoms, a halogen atom, hydroxyl, nitro, cyano, amino, carboxyl, a C₂₋₈ acyl group, or an alkoxycarbonyl group comprising a C₁₋₈ alkoxy moiety; and

each of R⁴ and R⁵ independently is hydrogen, a alkyl group, or a C₁₋₈ alkyl group having one to three halogen atoms.
(96) A compound having the formula (VIII) described in (95) or a pharmacologically acceptable salt thereof, wherein R is hydrogen or a C₁₋₈ alkyl group.
(97) A compound having the formula (VIII) described in (95) or a pharmacologically acceptable salt thereof, wherein R is hydrogen.
(98) A compound having the formula (VIII) described in (95) or a pharmacologically acceptable salt thereof, wherein R⁴ is hydrogen, and R⁵ is hydrogen or a C₁₋₈ alkyl group.
(99) A compound having the formula (VIII) described in (95) or a pharmacologically acceptable salt thereof, wherein each of R⁴ and R⁵ is hydrogen.
(100) A compound having the formula (VIII) described in (95) or a pharmacologically acceptable salt thereof, wherein R² is a C₁₋₈ alkoxy group, hydroxyl, carboxyl, cyano, or an alkoxycarbonyl group comprising a C₁₋₈ alkoxy moiety.
(101) A compound having the formula (VIII) described in (95) or a pharmacologically acceptable salt thereof, wherein R² is a C₁₋₈ alkoxy group or hydroxyl.
(102) A compound having the formula (VIII) described in (95) or a pharmacologically acceptable salt thereof, wherein R³ is hydrogen.
(103) A compound having the following formula (IX) or a pharmacologically acceptable salt thereof:

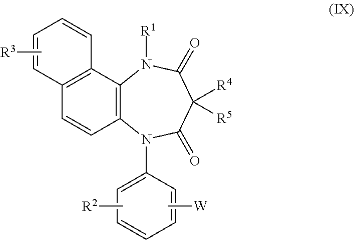

wherein R¹ is hydrogen, a C₁₋₈ alkyl group, a C₂₋₈ alkenyl group, a C₁₋₈ alkyl group having one to three halogen atoms, or a C₁₋₃ alkyl group having phenyl;

each of R² and R² independently is hydrogen, a C₁₋₉ alkyl group, a C₁₋₈ alkoxy group, a C₁₋₈ alkyl group having one to three halogen atoms, a C₁₋₈ alkoxy group having one to three halogen atoms, a halogen atom, hydroxyl, nitro, cyano, amino, a C₁₋₈ alkylamino group, a C₂₋₈ dialkylamino group, a C₂₋₈ acylamino group, a C₂₋₈ acylamino group having one to three halogen atoms, a C₁₋₈ alkylsulfonylamino group, carboxyl, a C₂₋₈ acyl group, an alkoxycarbonyl group comprising a C₁₋₈ alkoxy moiety, carbamoyl, a C₁₋₈ alkylthio group, a C₁₋₈ alkylsulfinyl group, a C₁₋₈ alkyl-sulfonyl group, or sulfamoyl;

each of R⁴ and R⁵ independently is hydrogen, a C₁₋₈ alkyl group, a C₁₋₈ alkyl group having one to three halogen atoms, or a C₁₋₃ alkyl group having phenyl; and

W is a five-membered or six-membered heterocyclic ring optionally having one or more substituents and comprising one to four nitrogen atoms as the members of the ring.
(104) A compound having the formula (IX) described in (103) or a pharmacologically acceptable salt thereof, wherein W is tetrazole, 1,2,4-triazole, 1,2,3-triazole, 1,2,4-oxadiazole, pyrazole, or imidazole, each of which optionally has one or more substituents selected from the group consisting of a C₁₋₈ alkyl group, a C₁₋₈ alkyl group having one to three halogen atoms, a halogen atom, cyano, oxo, and thioxo.
(105) A compound having the formula (IX) described in (103) or a pharmacologically acceptable salt thereof, wherein W is tetrazole, 1,2,4-triazole, or 1,2,3-triazole, each of which optionally has one or more substituents selected from the group consisting of a C₁₋₈ alkyl group, a alkyl group having one to three halogen atoms, a halogen atom, and cyano.
(106) A compound having the formula (IX) described in (103) or a pharmacologically acceptable salt thereof, wherein W is 5-oxo-1,2,4-oxadiazole or 5-thioxo-1,2,4-oxadiazole.
(107) A compound having the formula (IX) described in (103) or a pharmacologically acceptable salt thereof, wherein W is tetrazole.
(108) A compound having the formula (IX) described in (103) or a pharmacologically acceptable salt thereof, wherein R¹ is hydrogen or a C₁₋₈ alkyl group.
(109) A compound having the formula (IX) described in (103) or a pharmacologically acceptable salt thereof, wherein R¹ is hydrogen.
(110) A compound having the formula (IX) described in (103) or a pharmacologically acceptable salt thereof, wherein R⁴ is hydrogen, and R⁵ is hydrogen or a C₁₋₈ alkyl group.
(111) A compound having the formula (IX) described in (103) or a pharmacologically acceptable salt thereof, wherein each of R⁴ and is hydrogen.
(112) A compound having the formula (IX) described in (103) or a pharmacologically acceptable salt thereof, wherein R² is hydrogen, a C₁₋₈ alkyl group, a C₁₋₈ alkoxy group, a C₁₋₈ alkyl group having one to three halogen atoms, a C₁₋₈ alkoxy group having one to three halogen atoms, a halogen atom, hydroxyl, nitro, cyano, amino, carboxyl, a C₂₋₈ acyl group, or an alkoxycarbonyl group comprising a C₁₋₈ alkoxy moiety.
(113) A compound having the formula (IX) described in (103) or a pharmacologically acceptable salt thereof, wherein R² is hydrogen.
(114) A compound having the formula (IX) described in (103) or a pharmacologically acceptable salt thereof, wherein R¹ is hydrogen, a C₁₋₈ alkyl group, a C₁₋₈ alkoxy group, a C₁₋₈ alkyl group having one to three halogen atoms, a C₁₋₈ alkoxy group having one to three halogen atoms, a halogen atom, hydroxyl, nitro, cyano, amino, carboxyl, a C₂₋₈ acyl group, or an alkoxycarbonyl group comprising a C₁₋₈ alkoxy moiety.
(115) A compound having the formula (IX) described in (103) or a pharmacologically acceptable salt thereof, wherein R is hydrogen.
(116) 5-[3-(1H-tetrazol-5-yl)phenyl]-1H-naphtho[1,2-b][1,4]diazepine-2,4(3H,5H)-dione potassium salt.
(117) Paroxetine or a pharmacologically acceptable salt thereof.

The above-mentioned compounds can be prepared according to known processes. For example, the compounds described in (2) to (4) can be prepared according to a process described in WO 2004/085440. The compounds described in (5) and (117) can be prepared according to a process described in Japanese Patent Publication No. 59(1984)-48826. The compounds described in (8) to (16) can be prepared according to a process described in WO 2007/072974. The compounds described in (17) to (36) can be prepared according to a process described in WO 2007/074970. The compounds described in (37) to (52) can be prepared according to a process described in WO 2008/023847. The compounds described in (53) to (79) can be prepared according to a process described in WO 2009/022730. The compounds described in (80) to (94) can be prepared according to a process described in WO 2009/022731. The compounds described in (95) to (102) can be prepared according to a process described in WO 2010/090300. The compounds described in (103) to (116) can be prepared according to a process described in WO 2010/093061.

The compounds described in (7) and (117) such as paroxetine, imipramine are known compounds. The chemical structures and the documents disclosing the processes for preparation of the compounds are described in The MERCK INDEX FOURTEENTH EDITION (2006) or the like. Further, these compounds are commercially available.

The selective serotonin reuptake inhibitors described in (6) include paroxetine, fluoxetine, fluvoxamine, and citalopram.

The above-mentioned WO 2004/085440, WO 2007/072974, WO 2007/074970, WO 2008/023847, WO 2009/022730, WO 2009/022731, WO 2010/090300, WO 2010/093061, WO 2007/049825, and WO 2008/020651 describe that the compounds described in (2) to (117) have P2X₄ receptor antagonism.

The pharmacologically acceptable salts in the active ingredients of the present invention include a salt with an acid (e.g., hydrochloric acid, acetic acid, benzoic acid, fumaric acid, besylic acid), an alkali metal (e.g., sodium, potassium, lithium), or an amine.

The active ingredients of the present invention can be a geometrical (cis-trans) isomer or an optical isomer such as an optically active substance and racemic modification, each of which is included within the scope of the invention.

Hydrates can also be used as the active ingredients of the present invention.

The results of the pharmacological experiments are described below.

The effect of P2X₄ receptor antagonist on neuropathic pain was examined using an experimental autoimmune neuritis (FAN) rat model (Examples 3 and 4), which has been used as an experimental model for Guillain-Barré syndrome (GBS).

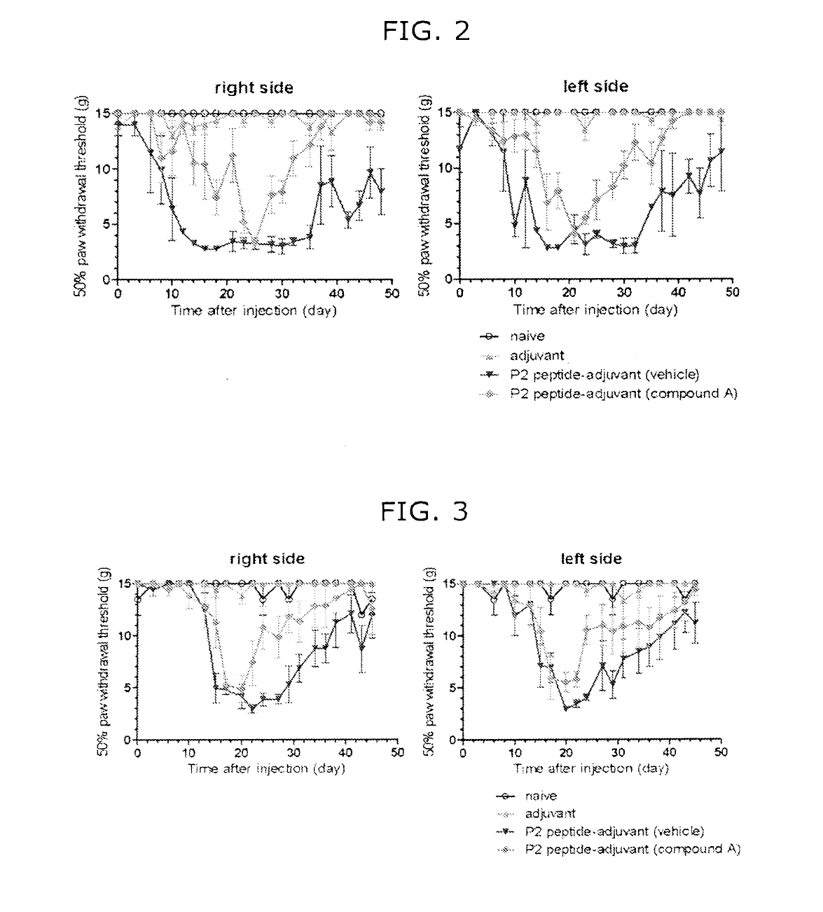

The results of Examples 3 and 4 as well as FIGS. 2 and 3 show analgesic activities of the compound A, which has P2X₄ receptor antagonism, on neuropathic pain originated from EAN. The results suggest that P2X₄ receptor plays a major role in neuropathic pain associated with Guillain-Barré syndrome.

Further, it is suggested using the EAN rat model in the acute phase of autoimmune neuritis that spinal microglial cells proliferate and proliferation and activation of expression of P2X₄ receptor play important roles in causing the GBS neuropathic pain. Therefore, it is furthermore indicated that P2X4 receptor antagonist can be an effective therapeutic agent for the GBS neuropathic pain.

The preventive or therapeutic agent of the present invention can be administered to human beings by ordinary administration methods such as oral administration or parenteral administration.

The compound can be granulated in ordinary manners for the preparation of pharmaceuticals. For instance, the compound can be processed to give tablets, granule, powder, capsule, suspension, injection, suppository, and the like.

Ordinary additives such as vehicles, disintegrators, binders, lubricants, and dyes are used for the preparation of these pharmaceuticals such as tablets. As the vehicles, lactose, D-mannitol, crystalline cellulose, and glucose can be mentioned. Further, there can be mentioned starch and carboxymethylcellulose calcium (CMC-Ca) as the disintegrators, magnesium stearate and talc as the lubricants, and hydroxylpropylcellulose (HPC), gelatin and polyvinylpyrrolidone (PVP) as the binders. The preparation of an injection can be made using solvents, stabilizers, dissolution-aids, suspensions, emulsifiers, soothing agents, buffers, or preservatives.

The compound of the invention can be administered to an adult generally in an amount of approximately 0.01 mg to 100 mg a day by parenteral administration and 1 mg to 2,000 mg a day by oral administration. The dosage can be adjusted in consideration of age and conditions of the patient.

### EXAMPLES

### Example 1

### (Experimental Procedure)

P2X₄ receptor antagonisms of the compound A (5-[3-(1H-tetrazol-5-yl)phenyl]-1H-naphtho[1,2-b][1,4]diazepine-2,4(3H,5H)-dione potassium salt and paroxetine were measured as described below.

ATP receptors (human P2X₄) were introduced into 1321N1 cells, and used as a stable ATP receptor-expressing system. The obtained P2X₄ expressing 1321N1 cells were plated in a 96-well assay plate, and cultured 24 hours at 37° C. in an atmosphere of 5% CO₂ for calcium assay. Fura-2 AM calcium fluorescent indicator was dissolved in an extracellular solution for calcium imaging. The obtained solution was loaded onto the plated cells, and placed at room temperature for 45 minutes to introduce Fura-2 AM into the cells. The fluorescence was detected by EnVision micro plate reader (PerkinElmer). The cells were alternatively illuminated with two excitations wavelengths (lights through 340 nm and 380 nm filters) via xenon lamp, and the emitted fluorescence was measured at 510 nm. The fluorescence changes were monitored to determine the fluorescence ratio (F340/F380) as the index of intracellular calcium change. Measurements were conducted by adding 1 μM ATP to each well, and monitoring the ATP induced intracellular calcium responses with the passage of time. Tested compounds were treated to cells 15 min before the addition of ATP, and the inhibitory activities of compounds were calculated by comparing the calcium response with control in the absence of tested compound.

### (Experimental Results)

TABLE 1

| Test compound | IC₅₀ (μM) |
|---|---|
| Paroxetine | 4.6 |
| Compound A | 0.27 |

### Example 2

Proliferation of spinal microglial cells and increasing of expression of P2X₄ receptor in the acute phase of autoimmune neuritis were researched by immunohistological analysis using the EAN rat (Reiter et al.: J. Neuroimmunol. 2005 March; 160(1-2):25-31).

### (Experimental Procedure)

Nine-week-old male LEW/CrlCrlj rat was anesthetized with isoflurane, and an adjuvant or P2 peptide-adjuvant solution was administered by intradermal tale base injection in an amount of 80 μpg/80 μL/rat to obtain the EAN rat model. The P2 peptide-adjuvant solution was prepared by dissolving neuritogenic P2 peptide of peripheral myelin (amino acids 53-78: TESPFKNTEISFKLGQEFEETTADNR) in PBS, and mixing the obtained 2 mg/mL solution with complete Freund's adjuvant containing 2 mg/mL (the same concentration) of mycobacterium tuberculosis.

Eighteen days after immunization, the spinal cord was collected after perfusion of 41 neutral buffered paraformaldehyde, embedded with paraffin to prepare slices. A specimen in cross section was prepared at the fifth lumbar level (L5) of the spinal cord, and was subjected to an immunohistological staining using Iba1 antibody, which has widely been used as a microglia marker, and P2X₂ receptor antibody.

### (Experimental Results)

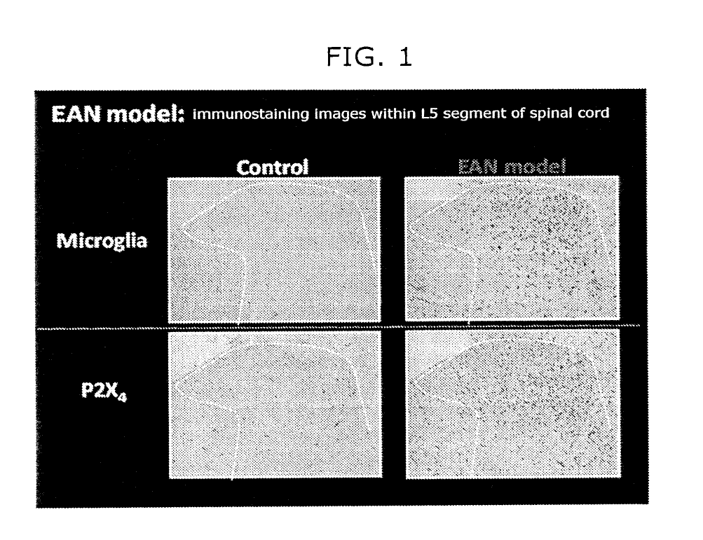

The obtained immunostaining images are shown in FIG. 1. It is observed that Iba1 (antigen specific to microglia)-positive cell signals (upper figures) and P2X₄ receptor-positive signals (lower figures) increase within L5 segment of the spinal cord, compared with the sides administered with only adjuvant.

### Example 3

### (Experimental Procedure)

Six-week-old male LEW/CrlCrlj rat was acclimatized for about one week, and an indwelling polystyrene catheter with a 0.30 mm outside diameter was placed into the subarachnoid space. Three days or more after indwelling of the catheter for administration into the subarachnoid space, the rat was anesthetized with isoflurane, and an adjuvant or P2 peptide-adjuvant solution was administered by intradermal tale base injection in an amount of 80 μg/80 μL/rat. The compound A was continuously administered by Micro Infusion Pump (Primetech). The pump was placed at the same time of administration of P2 peptide-adjuvant. Administration of the compound A solution was started while placing the pump. After immunization, change of pain threshold was observed with the passage of time.

### (Experimental Results)

FIG. 2 shows influence of preventive administration of the compound A on pain threshold of FAN rat model. The animal was administered with P2 peptide, and neuritis associated with paresis of hind legs was observed about ten days after immunization. Further, allodynia was simultaneously observed. Thereafter, allodynia was continued for about 50 days. The animal model was preventively administered with the compound A to suppress pains in initial and later manifestations of the disease.

### Example 4

### (Experimental Procedure)

Six-week-old male LEW/CrlCrlj rat was acclimatized for about one week, and an indwelling polystyrene catheter with a 0.30 mm outside diameter was placed into the subarachnoid space. Three days or more after indwelling of the catheter for administration into the subarachnoid space, the rat was anesthetized with isoflurane, and an adjuvant or P2 peptide-adjuvant solution was administered by intradermal tale base injection in an amount of 80 μg/80 μL/rat. Micro Infusion Pump was simultaneously placed into the back of the rat, and administration of the vehicle into the subarachnoid space was initiated. After immunization, symptom was observed (Table 2), and change of pain threshold was observed. Thirteen days after immunization, the average of manifestation scores rose up to 2 or more, and they were divided into groups to observe influence of therapeutic administration of the compound A on pain threshold.

### (Experimental Results)

FIG. 3 shows influence of therapeutic administration of the compound A on pain threshold of EAN rat model.

A significant analgesic effect on pain in later manifestation of the disease was observed in therapeutic administration as well as the preventive administration.

TABLE 2
Scores of symptom observation

| | |
|---|---|
| Score 0 | Normal |
| Score 1 | Reduced tone of the tail |
| Score 2 | Limp tail |
| Score 3 | Gate ataxia |
| Score 4 | Hemiplegia of the hind leg |
| Score 5 | Paraplegia of the hind legs |
| Score 6 | Tetraparesis |
| Score 7 | Moribond |
| Score 8 | Death |

What is claimed is:

1. A method of preventing or treating neuropathic pain associated with Guillain-Barré syndrome comprising administrating an effective amount of P2X₄ receptor antagonist or a selective serotonin reuptake inhibitor to a patient in need thereof.
